(19) United States
(12) Patent Application Publication
(10) Pub. No.: US 2026/0283921 A1
(43) Pub. Date: Sep. 24, 2026

(54) SUNSCREEN WITH DISODIUM PHENYL DIBENZIMIDAZOLE TETRASULFONATE AND TEREPHTHALYLIDENE DICAMPHOR SULFONIC ACID

(71) Applicant: BEIERSDORF AG, Hamburg (DE)

(72) Inventors: David SCHLENKER, Hamburg (DE); Laetitia NEUMANN, Welle (DE); Carsten HEINSEN, Barmstedt (DE)

(21) Appl. No.: 19/469,060

(22) PCT Filed: Mar. 15, 2024
(86) PCT No.: PCT/EP2024/056944

§ 371 Date: Sep. 25, 2025

(30) Foreign Application Priority Data
Mar. 30, 2023 (DE) 10 2023 202 921.9

(51) Int. Cl. A61K 8/49; A61K 8/46; A61Q 17/04

(52) U.S. Cl. CPC A61K 8/4946; A61K 8/466; A61Q 17/04; A61K 2800/30

(57) ABSTRACT

Cosmetic preparation containing a) the triethanolamine salt of disodium phenyl dibenzimidazole tetrasulfonate (INCI: Disodium Phenyl Dibenzimidazole Tetrasulfonate), b) the triethanolamine salt of terephthalylidene dicamphor sulfonic acid (INCI: Terephthalylidene Dicamphor Sulfonic Acid).

The present invention relates to a cosmetic preparation comprising the triethanolamine salt of disodium phenyl dibenzimidazole tetrasulfonate (INCI: Disodium Phenyl Dibenzimidazole Tetrasulfonate) and the triethanolamine salt of terephthalylidene dicamphor sulfonic acid (INCI:

### Terephthalylidene Dicamphor Sulfonic Acid).

For many years there has been an unbroken trend away from aristocratic pallor toward “healthy, athletically brown skin”. To achieve this, people expose their skin to solar radiation, since this causes pigment formation in the sense of melanin formation. However, the ultraviolet radiation of sunlight also has a harmful effect on the skin. Besides acute damage (sunburn), long-term damage such as an increased risk of developing skin cancer occurs in the case of excessive irradiation with light from the UVB range (wavelength: 280-320 nm). Excessive exposure to UVB and UVA radiation (wavelength: 320-400 nm) additionally leads to weakening of the elastic and collagen fibers in connective tissue. This leads to numerous phototoxic and photoallergic reactions, resulting in premature aging of the skin.

Therefore, to protect the skin, a range of photoprotective filter substances that can be used in cosmetic preparations have been developed. In most industrialized countries, these UVA and UVB filters are collated in the form of positive lists such as Annex 7 of the Kosmetikverordnung

### [German Cosmetics Ordinance].

The multiplicity of commercially available sunscreens must not, however, distract from the fact that these prior-art preparations have a range of disadvantages.

For many years there has been a constantly growing demand for UV-A filter substances. This can be attributed not least to numerous government regulations that require sunscreens to have a balanced ratio of UV-A and UV-B protection. However, the number of UV-A filters that can be used in cosmetics is very limited. Use is essentially made of oil-soluble compounds such as Butyl Methoxydibenzoylmethane or Diethylamino Hydroxybenzoyl Hexyl Benzoate.

In order to achieve particularly high UV protection, it is necessary, however, to also incorporate UV-A filters into the aqueous phase. For this purpose, around the turn of the millennium, Symrise developed the compound disodium phenyl dibenzimidazole tetrasulfonate (INCI: Disodium Phenyl Dibenzimidazole Tetrasulfonate) as a water-soluble UV-A filter. However, this substance has the disadvantage of fluorescing strongly in certain wavelength ranges. This effect was first observed by visitors in nightclubs who sparkled in a bluish manner under “blacklight”. This fluorescence phenomenon initially made the UV filter unusable for further uses in cosmetics and a number of research projects were launched in order to remedy the problem. Symrise found a first promising approach in the use of troxerutin. This solution was successfully patented with EP2185126.

Although the combination with troxerutin led to a significant reduction in the fluorescence, a weak residual fluorescence still remained, which further hindered the use of Disodium Phenyl Dibenzimidazole Tetrasulfonate in cosmetics. There are also reasons to avoid troxerutin in view of the costs of such a preparation and the compatibility thereof (in individual cases allergic reactions cannot be ruled out).

It was therefore the object of the present invention to solve this problem of the prior art and to further reduce the fluorescence of disodium phenyl dibenzimidazole tetrasulfonate (INCI:

Disodium Phenyl Dibenzimidazole Tetrasulfonate) in cosmetic preparations without having to use troxerutin, or to reduce the use amount thereof.

Surprisingly, the object is achieved by a cosmetic preparation comprising
a) the triethanolamine salt of disodium phenyl dibenzimidazole tetrasulfonate (INCI: Disodium Phenyl Dibenzimidazole Tetrasulfonate),
b) the triethanolamine salt of terephthalylidene dicamphor sulfonic acid (INCI: Terephthalylidene Dicamphor Sulfonic Acid).

Those skilled in the art are familiar with EP2185126. In addition, there is DE 10 2022 203499.6, which was not yet published at the time of the present application, but these documents were unable to point the way to the present invention.

It is advantageous according to the invention if the preparation according to the invention comprises disodium phenyl dibenzimidazole tetrasulfonate (INCI: Disodium Phenyl Dibenzimidazole Tetrasulfonate) in a concentration of 0.1% to 5% by weight, based on the total weight of the preparation. A content from 0.5% to 3% by weight, based on the total weight of the preparation, is preferred here according to the invention.

This compound is neutralized with triethanolamine during the production of the preparation.

Moreover, the embodiments of the present invention that are advantageous according to the invention are characterized in that the preparation comprises terephthalylidene dicamphor sulfonic acid (INCI: Terephthalylidene Dicamphor Sulfonic Acid) in a concentration of 0.1% to 3% by weight, based on the total weight of the preparation. A content from 1% to 3% by weight, based on the total weight of the preparation, is preferred here according to the invention.

This sulfonic acid is also neutralized with triethanolamine during the production of the preparation.

Furthermore, it is advantageous in the context of the present invention if the weight ratio of disodium phenyl dibenzimidazole tetrasulfonate (INCI: Disodium Phenyl Dibenzimidazole Tetrasulfonate) to terephthalylidene dicamphor sulfonic acid (INCI: Terephthalylidene Dicamphor Sulfonic Acid) is from 0:3 to 1:1.

It is advantageous according to the invention if the preparation according to the invention is in the form of an aqueous or aqueous-alcoholic gel.

If the product is an aqueous gel, then the aqueous phase of the neutralized disodium phenyl dibenzimidazole tetrasulfonate is incorporated into the neutralized aqueous phase of the terephthalylidene dicamphor sulfonic acid (INCI: Terephthalylidene Dicamphor Sulfonic Acid). In other words: two aqueous subphases, each with one of the sulfonic acids, are prepared, neutralized separately with triethanolamine and then combined to form an aqueous preparation.

Embodiments of the present invention that are advantageous according to the invention are characterized in that the preparation is free of 2-ethylhexyl 2-cyano-3,3-diphenylacrylate (Octocrylene), homomenthyl salicylate (Homosalate), 2-ethylhexyl 4-methoxycinnamate, 3-(4-methylbenzylidene) camphor, 2-hydroxy-4-methoxybenzophenone (Oxybenzone), methyl anthranilate (INCI: Methyl Anthranilate) and sulisobenzone (INCI: Benzophenone-4).

Moreover, it is advantageous according to the invention if the preparation according to the invention is free of polyethylene glycol, polyethylene glycol ethers and polyethylene glycol esters (so-called PEG derivatives), is free of polyacrylates, mineral oils and silicone oils, and is free of parabens, methylisothiazolinone, chloromethylisothiazolinone and DMDM hydantoin.

For the cosmetic preparation according to the invention or the process according to the invention, it is particularly advantageous according to the invention if the preparation comprises 2-phenylbenzimidazole-5-sulfonic acid salts.

In such a case, the embodiments that are preferred according to the invention are characterized in that the preparation comprises 2-phenylbenzimidazole-5-sulfonic acid salts in a concentration of 0.1% to 8% by weight, based on the total weight of the preparation.

It is also advantageous according to the invention if the sodium salt or the triethanolamine salt is used as 2-phenylbenzimidazole-5-sulfonic acid salts.

It is advantageous according to the invention if the preparation comprises one or more compounds selected from the group of xanthan gum (INCI: Xanthan Gum), welan gum (INCI: Welan Gum), gellan gum (INCI: Gellan Gum), sclerotium gum (INCI: Scerotium Gum), cellulose and/or cellulose derivatives. Advantageous celluloses and cellulose derivatives include for example microcrystalline cellulose, ethyl cellulose, propyl cellulose, hydroxypropyl cellulose, methoxyethyl cellulose, methoxypropyl cellulose and carboxymethyl cellulose.

Furthermore, it is particularly advantageous according to the invention if the preparation according to the invention comprises ethylhexylglycerin and/or 4-hydroxyacetophenone.

If the preparation according to the invention comprises only ethylhexylglycerin and no 4-hydroxyacetophenone, then the use concentration that is advantageous according to the invention is from 0.1% to 0.5% by weight, based on the total weight of the preparation.

If the preparation according to the invention comprises only 4-hydroxyacetophenone and no ethylhexylglycerin, then the use concentration that is advantageous according to the invention is from 0.1% to 0.6% by weight, based on the total weight of the preparation.

If the preparation according to the invention comprises both ethylhexylglycerin and 4-hydroxyacetophenone, then the use concentration that is advantageous according to the invention for ethylhexylglycerin is from 0.1% to 0.5% by weight and for 4-hydroxyacetophenone is from 0.1% to 0.6% by weight, in each case based on the total weight of the preparation.

Furthermore, it is advantageous according to the invention if the preparation comprises silica and/or glass beads having a particle diameter of 5 to 120 μm.

Furthermore, if the preparation according to the invention comprises a lipophilic phase, it is advantageous according to the invention if the preparation or process is characterized in that the preparation comprises hexyl 2-(4′-diethylamino-2′-hydroxybenzoyl)benzoate, 2,4,6-tris [anilino-(p-carbo-2′-ethyl-1′-hexyloxy)]-1,3,5-triazine (INCI: Ethylhexyl Triazone) and/or 2,4-bis {[4-(2-ethylhexyloxy)-2-hydroxy]phenyl}-6-(4-methoxyphenyl)-1,3,5-triazine (INCI: Bis-Ethylhexyloxyphenol Methoxyphenyl Triazine).

In such a case, it is also advantageous according to the invention if the preparation or process is characterized in that the preparation comprises 2,4,6-tris(biphenyl-4-yl)-1,3,5-triazine (INCI: Tris-Biphenyl Triazine), 2-ethoxyethyl(2Z)-2-cyano-2-[3-(3-methoxypropylamino)cyclohex-2-en-1-ylidene] acetate (INCI: Methoxypropylamino Cyclohexenylidene Ethoxyethylcyanoacetate) and/or the compound

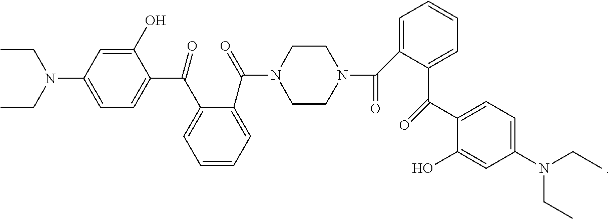 [SMILES: CCN(CC)c1ccc(C(=O)c2ccccc2C(=O)N2CCN(C(=O)c3ccccc3C(=O)c3ccc(N(CC)CC)cc3O)CC2)c(O)c1]

Furthermore, in such a case it is advantageous according to the invention if the preparation comprises lauryl glucoside and/or decyl glucoside.

Embodiments of the present invention that are advantageous according to the invention are characterized in that the preparation comprises one or more compounds selected from the following group of compounds: alpha-lipoic acid, folic acid, phytoene, D-biotin, coenzyme Q10, alpha-glucosylrutin, carnitine, carnosine, natural and/or synthetic isoflavonoids, flavonoids, creatine, creatinine, taurine, β-alanine, panthenol, magnolol, honokiol, tocopheryl acetate, dihydroxyacetone; 8-hexadecene-1,16-dicarboxylic acid, glycerylglucose, (2-hydroxyethyl) urea, vitamin E and derivatives thereof, hyaluronic acid and/or salts thereof, and/or licochalcone A.

It is advantageous according to the invention if the preparation comprises one or more alkanediols from the following group of compounds: pentane-1,2-diol, hexane-1,2-diol, octane-1,2-diol, decane-1,2-diol, 2-methylpropane-1,3-diol. The use of hexane-1,2-diol is preferred here according to the invention.

It is also advantageous in the context of the present invention if the preparation according to the invention comprises one or more perfumes selected from the following group of compounds: limonene, citral, linalool, alpha-isomethylionone, geraniol, citronellol, 2-isobutyl-4-hydroxy-4-methyltetrahydropyran, 2-tert-pentylcyclohexyl acetate, 3-methyl-5-phenyl-1-pentanol, 7-acetyl-1,1,3,4,4,6-hexamethyltetralin, alpha-amylcinnamaldehyde, alpha-methylionone, amyl C, butylphenyl methylpropional cinnamal, amyl salicylate, amylcinnamyl alcohol, anise alcohol, benzoin, benzyl alcohol, benzyl benzoate, benzyl cinnamate, benzyl salicylate, bergamot oil, bitter orange oil, butylphenyl methylpropional, cardamom oil, cedrol, cinnamal, cinnamyl alcohol, citronellyl methylcrotonate, lemon oil, coumarin, diethyl succinate, ethyl linalool, eugenol, Evernia furfuracea extract, Evernia prunastri extract, farnesol, guaiac wood oil, hexyl cinnamal, hexyl salicylate, hydroxycitronellal, lavender oil, lime oil, linalyl acetate, mandarin oil, menthyl PCA, methylheptenone, nutmeg oil, rosemary oil, sweet orange oil, terpineol, tonka bean oil, triethyl citrate and/or vanillin.

According to the invention, the preparation according to the invention advantageously comprises film formers. In the context of the present invention, film formers are substances of varying composition that are characterized by the following property: if a film former is dissolved in water or other suitable solvents and the solution is then applied to the skin, then, after the solvent has evaporated, it forms a film that essentially serves to fix the light filters on the skin and thus to increase the water resistance of the product.

These film formers may for example be selected from the group of polymers based on polyvinylpyrrolidone (PVP) or are copolymers of polyvinylpyrrolidone, for example PVP Hexadecene Copolymer and PVP Eicosene Copolymer, which are available under the trade names Antaron V216 and Antaron V220 from GAF Chemicals Corporation.

However, it is preferred according to the invention if maize starch, maltodextrin and/or pullulan are used for this purpose.

Last but not least, the preparations and processes according to the invention are characterized in that the preparation is free of troxerutin.

### Comparative Experiment

The following formulations were produced and the fluorescence properties thereof were investigated:

| | 104 | 105 | 106 | 107 | 102 | 103 |
|---|---|---|---|---|---|---|
| EU INCI | % by | % by | % by | % by | % by | % by |
| Declaration | weight | weight | weight | weight | weight | weight |
| Ethylhexylglycerin | 0.20 | 0.20 | 0.20 | 0.20 | 0 | 0 |
| Hexane-1,2-diol | 0.35 | 0.35 | 0.35 | 0.35 | 0 | 0 |
| Aqua + Sodium | 2.62 | 1.77 | 0 | 0 | q.s. | 0 |
| Hydroxide | | | | | | |
| Triethanolamine + | 0 | 0 | 5.85 | 4.26 | 0 | q.s. |
| Aqua | | | | | | |
| Phenoxyethanol + | 0.80 | 0.80 | 0.80 | 0.80 | 0 | 0 |
| Aqua | | | | | | |
| Aqua | 82.53 | 91.38 | 79.30 | 88.89 | to 100 | to 100 |
| Aqua + Trisodium | 0.50 | 0.50 | 0.50 | 0.50 | 0 | 0 |
| EDTA | | | | | | |
| | 8.00 | 0 | 8.00 | 0 | | |
| Disodium | 2.00 | 2.00 | 2.00 | 2.00 | 3 | 3 |
| Phenyl | | | | | | |
| Dibenzimidazole | | | | | | |
| Tetrasulfonate | | | | | | |
| Phenylbenz- | 3.00 | 3.00 | 3.00 | 3.00 | 0 | 0 |
| imidazole | | | | | | |
| Sulfonic Acid | | | | | | |
| Total: | 100.00 | 100.00 | 100.00 | 100.00 | 100.00 | 100.00 |
| Blue value at | 0.073 ± 0.013 | 0.166 ± 0.011 | 0.05 ± 0.008 | 0.12 ± 0.007 | 0.566 ± 0.022 | 0.437 ± 0.029 |
| 365 nm | | | | | | |
| Blue value at | 0.076 ± 0.013 | 0.185 ± 0.008 | 0.06 ± 0.006 | 0.145 ± 0.01 | 0.547 ± 0.021 | 0.357 ± 0.03 |
| 312 nm | | | | | | |

The fluorescence (color intensity at 365 and 312 nm) was determined as follows:

3.0 ml of the samples are first transferred into glass Petri dishes (diameter 5 cm). The Petri dishes are then placed individually on a dark surface. A commercially available DSLR or DSLM camera (here a Canon PowerShot SX540 HS) is mounted at a height of 30 cm. A UV lamp (here a VL-6.LM 365/312 2×6W UV lamp 230V EU; set to 365 or 312 nm) is mounted in the same manner. The images in these examples were created with the following camera settings and are for guidance purposes only: ISO 400; F=4.0; self-timer 25 s. The images are evaluated in a program that can separate them into color channels. GIMP 2.10.28 was used in the present case. The color channels of the images can be separated into RGB colors using a histogram. The evaluated area of the images must be selected appropriately so that only the contents of the Petri dishes are separated in the histogram. The value for the fluorescence is determined on the basis of the blue value at 365 or 312 nm excitation wavelength.

Conclusion: The fluorescence and the blue value are reduced by the presence of terephthalylidene dicamphor sulfonic acid and by the presence of triethanolamine.

### EXAMPLES

The examples that follow are intended to illustrate the present invention without limiting it. Unless stated otherwise, all amounts, proportions and percentages are based on the weight and the total amount or on the total weight of the preparations.

| | Sample | Sample | Sample | Sample | Sample | Sample | Sample | Sample | Sample | Sample |
|---|---|---|---|---|---|---|---|---|---|---|
| Raw Material | 1 | 2 | 3 | 4 | 5 | 6 | 7 | 8 | 9 | 10 |
| Disodium Phenyl | 2 | 1 | 2 | 1 | 2 | 2 | 2 | 0 | 2 | 2 |
| Dibenzimidazole | | | | | | | | | | |
| Tetrasulfonate | | | | | | | | | | |
| Phenylbenzimidazole | 4 | 3 | 3 | 2 | 4 | 3 | 3 | 2 | 3 | 3 |
| Sulfonic Acid | | | | | | | | | | |
| Terephthalylidene | 3 | 3 | 3 | 3 | 2.5 | 2.5 | 2 | 3 | 3 | 2.5 |
| Dicamphor Sulfonic | | | | | | | | | | |
| Acid | | | | | | | | | | |
| Glycerin | 10 | 0 | 5 | 0 | 2 | 0 | 2 | 2 | 5 | 5 |
| VP/Hexadecene | 0 | 0 | 0 | 0 | 0 | 0 | 0 | 0 | 0 | 0.5 |
| Copolymer | | | | | | | | | | |
| Glyceryl Stearate | 0 | 0 | 0 | 0 | 0 | 0 | 0 | 0 | 0 | 1 |
| Triethanolamine | 2.6 | 2.1 | 2.6 | 2.1 | 2.35 | 2.35 | 2.1 | 1.6 | 2.6 | 2.35 |
| Sodium Hydroxide | 0.6 | 0.45 | 0.45 | 0.3 | 0.6 | 0.45 | 0.45 | 0.3 | 0.45 | 0.45 |
| Xanthan Gum | 1 | 2 | 1 | 1 | 2 | 1 | 1 | 0.5 | 1 | 2 |
| Ethylhexylglycerin | 0.2 | 0.2 | 0.2 | 0 | 0.1 | 0.2 | 0.2 | 0.2 | 0.2 | 0.2 |
| Trisodium EDTA | 0.5 | 0.5 | 0.5 | 0.2 | 0.5 | 1 | 0.5 | 0.7 | 0.5 | 0.5 |
| Phenoxetol | 0.3 | 0.5 | 0.8 | 0 | 0.2 | 0.5 | 0.5 | 0.5 | 0.5 | 0.5 |
| Hexane-1,2-diol | 0.35 | 0.25 | 0.35 | 0 | 0.15 | 0.25 | 0.35 | 0.25 | 0.25 | 0.25 |
| Ethanol | 0 | 0 | 0 | 15 | 5 | 5 | 0 | 0 | 0 | 5 |
| Pullulan | 0 | 0 | 0 | 0 | 0.2 | 0 | 0 | 0 | 0.5 | 0 |
| Polyglyceryl-10 | 0 | 0 | 0 | 0 | 0 | 0 | 0 | 0 | 0 | 1 |
| Stearate | | | | | | | | | | |
| Tris-Biphenyl Triazine | 0 | 0 | 0 | 0 | 0 | 0 | 0 | 0 | 0 | 1 |
| (and) Aqua (and) | | | | | | | | | | |
| Decyl Glucoside (and) | | | | | | | | | | |
| Butylene Glycol (and) | | | | | | | | | | |
| Disodium Phosphate | | | | | | | | | | |
| (and) Xanthan Gum | | | | | | | | | | |
| Glass Beads | 0 | 0 | 0 | 1 | 0 | 0 | 1 | 1 | 0 | 1 |
| C12-15 Alkyl | 0 | 0 | 0 | 0 | 0 | 0 | 0 | 0 | 0 | 4 |
| Benzoate | | | | | | | | | | |
| Ethylhexyl Triazone | 0 | 0 | 0 | 0 | 0 | 0 | 0 | 0 | 0 | 2 |
| Aqua | Add to | Add to | Add to | Add to | Add to | Add to | Add to | Add to | Add to | Add to |
| | 100 | 100 | 100 | 100 | 100 | 100 | 100 | 100 | 100 | 100 |

1.-16. (canceled)

17. A cosmetic preparation, wherein the preparation comprises (a) triethanolamine salt of disodium phenyl dibenzimidazole tetrasulfonate and (b) triethanolamine salt of terephthalylidene dicamphor sulfonic acid.

18. The preparation of claim 17, wherein the preparation comprises disodium phenyl dibenzimidazole tetrasulfonate in a concentration of from 0.1% to 5% by weight, based on a total weight of the preparation.

19. The preparation of claim 17, wherein the preparation comprises terephthalylidene dicamphor sulfonic acid in a concentration of from 0.1% to 3% by weight, based on a total weight of the preparation.

20. The preparation of claim 18, wherein the preparation comprises terephthalylidene dicamphor sulfonic acid in a concentration of from 0.1% to 3% by weight, based on a total weight of the preparation.

21. The preparation of claim 17, wherein a weight ratio of disodium phenyl dibenzimidazole tetrasulfonate to terephthalylidene dicamphor sulfonic acid is from 0:3 to 1:1.

22. The preparation of claim 17, wherein the preparation is in the form of an aqueous or aqueous-alcoholic gel.

23. The preparation of claim 17, wherein the preparation is free of 2-ethylhexyl 2-cyano-3,3-diphenylacrylate (Octocrylene), homomenthyl salicylate (Homosalate), 2-ethylhexyl 4-methoxycinnamate, 3-(4-methylbenzylidene)-camphor, 2-hydroxy-4-methoxybenzophenone (Oxybenzone), methyl anthranilate and sulisobenzone (Benzophenone-4).

24. The preparation of claim 17, wherein the preparation is free of polyethylene glycol, polyethylene glycol ethers, polyethylene glycol esters, polyacrylates, mineral oils, silicone oils, parabens, methylisothiazolinone, chloromethylisothiazolinone and DMDM hydantoin.

25. The preparation of claim 17, wherein the preparation comprises one or more 2-phenylbenzimidazole-5-sulfonic acid salts.

26. The preparation of claim 25, wherein the preparation comprises the one or more 2-phenylbenzimidazole-5-sulfonic acid salts in a concentration of from 0.1% to 8% by weight, based on a total weight of the preparation.

27. The preparation of claim 26, wherein the one or more salts of 2-phenylbenzimidazole-5-sulfonic acid comprise the sodium salt and/or the triethanolamine salt of 2-phenylbenzimidazole-5-sulfonic acid.

28. The preparation of claim 17, wherein the preparation further comprises one or more of xanthan gum, welan gum, gellan gum, sclerotium gum, cellulose, a cellulose derivative.

29. The preparation of claim 17, wherein the preparation further comprises ethylhexylglycerin.

30. The preparation of claim 17, wherein the preparation further comprises 4-hydroxyacetophenone.

31. The preparation of claim 17, wherein the preparation further comprises silica.

32. The preparation of claim 17, wherein the preparation further comprises glass beads having a particle diameter of from 5 to 120 μm.

33. The preparation of claim 17, wherein preparation further comprises one or more of maize starch, maltodextrin, pullulan.

34. The preparation of claim 17, wherein the preparation is free of troxerutin.

35. A process for producing the aqueous gel of claim 22, wherein the process comprises neutralizing terephthalylidene dicamphor sulfonic acid by triethanolamine in a first water phase and then combining it with a separately produced second aqueous phase in which disodium phenyl dibenzimidazole tetrasulfonate has been neutralized with triethanolamine.
